(19) United States
(12) Patent Application Publication
(10) Pub. No.: US 2026/0159312 A1
(43) Pub. Date: Jun. 11, 2026

(54) ONBOARD CONTAMINANT DETECTION SYSTEM

(71) Applicant: Oshkosh Corporation, Oshkosh, WI (US)

(72) Inventors: Jerrod Kappers, Oshkosh, WI (US); Vince Andrada, Oshkosh, WI (US); Joel Komurka, Oshkosh, WI (US); Joseph Wigle, Oshkosh, WI (US); Vince Schad, Oshkosh, WI (US); Brian Brost, Oshkosh, WI (US)

(73) Assignee: Oshkosh Corporation, Oshkosh, WI (US)

(21) Appl. No.: 19/413,356

(22) Filed: Dec. 9, 2025

Related U.S. Application Data
(60) Provisional application No. 63/730,192, filed on Dec. 10, 2024.

(51) Int. Cl. B65F 3/00; A62C 3/07; B65F 3/14; B65F 3/18; G01N 33/00

(52) U.S. Cl. CPC B65F 3/001; A62C 3/07; B65F 3/18; G01N 33/0078; B65F 2003/146

(57) ABSTRACT

A refuse vehicle may include a chassis, a body, a contaminant sensor, a conveyor, a contaminant separating mechanism, and a controller. The body is supported by the chassis and defines a refuse compartment and a containment volume. The contaminant sensor is disposed within the refuse compartment. The conveyor is disposed within the refuse compartment and configured to convey refuse material past the contaminant sensor. The contaminant separating mechanism is configured to selectively direct refuse material into the containment volume. The controller is configured to receive sensor data from the contaminant sensor. The controller is also configured to determine a presence of a contaminant within the refuse material based on the sensor data. The controller is also configured to actuate the contaminant separating mechanism in response to the presence of the contaminant to direct the refuse material containing the contaminant into the containment volume.

### CROSS-REFERENCE TO RELATED APPLICATIONS

The present application claims the benefit of and priority to U.S. Provisional Ser. No. 63/730,192, filed on Dec. 10, 2024, the entire contents of which are hereby incorporated by reference herein.

### BACKGROUND

The present disclosure generally relates to the field of refuse vehicles and systems for analyzing refuse materials received by the refuse vehicle.

### SUMMARY

An exemplary embodiment of the present disclosure relates to a refuse vehicle. The refuse vehicle includes a chassis, a body, a contaminant sensor, a conveyor, a contaminant separating mechanism, and a controller. The body is supported by the chassis and defines a refuse compartment and a containment volume. The contaminant sensor is disposed within the refuse compartment. The conveyor is disposed within the refuse compartment and configured to convey refuse material past the contaminant sensor. The contaminant separating mechanism is configured to selectively direct refuse material into the containment volume. The controller is configured to receive sensor data from the contaminant sensor. The controller is also configured to determine a presence of a contaminant within the refuse material based on the sensor data. The controller is also configured to actuate the contaminant separating mechanism in response to the presence of the contaminant to direct the refuse material containing the contaminant into the containment volume.

Another exemplary embodiment of the present disclosure relates to a contaminant detection system. The contaminant detection system includes a refuse body, a contaminant sensor, and a controller. The refuse body defines a storage volume. The contaminant sensor is configured to generate sensor data indicative of a contaminant within refuse deposited into the storage compartment. The controller is configured to receive sensor data from the contaminant sensor. The controller is also configured to determine a presence of a contaminant within refuse deposited into the storage compartment based on the sensor data. The controller is also configured to initiate remedial action in response to the presence of the contaminant to isolate or remove the contaminant from the storage compartment.

Another exemplary embodiment of the present disclosure relates to a method of detecting contaminants in refuse material within a refuse storage compartment. The method includes receiving, by a controller, sensor data from a contaminant sensor. The method also includes determining, by the controller based on the sensor data, that a contaminant is present in the refuse material. The method also includes initiating, by the controller in response to the presence of the contaminant, remedial action to at least one of isolate the contaminant or remove the contaminant from the refuse storage compartment.

Another exemplary embodiment of the present disclosure relates to a refuse vehicle. The refuse vehicle includes a chassis, a cab coupled to the chassis, a body supported by the chassis, a first conveyor, and a first contaminant sensor. The body defines a refuse compartment. The first conveyor is disposed within the refuse compartment and positioned to convey refuse received at a first position within the refuse compartment to a second position within the refuse compartment. The first contaminant sensor is disposed within the refuse compartment and oriented toward the first conveyor. The first contaminant sensor is configured to generate sensor data indicative of contaminants in refuse material on the first conveyor.

In some embodiments the body includes a cover, and the first contaminant sensor is coupled to an inside surface of the cover. In some embodiments, the first contaminant sensor is a camera. In some embodiments, the first contaminant sensor is a spectral imaging sensor. In some embodiments, the first contaminant sensor is a particle imaging sensor. In some embodiments, the first contaminant sensor is a magnetic field sensor. In some embodiments, the refuse vehicle includes a second contaminant sensor disposed within the refuse compartment, oriented toward the first conveyor, and configured to generate sensor data indicative of contaminants in refuse material on the first conveyor. In some embodiments, the second contaminant sensor is of a different type than the first contaminant sensor. In some embodiments, the refuse vehicle includes a second conveyor disposed within the refuse compartment and positioned to convey refuse conveyed by the first conveyor from the second position within the refuse compartment to a third position within the refuse compartment. In some embodiments, the refuse vehicle includes a second contaminant sensor disposed within the refuse compartment, oriented toward the second conveyor, and configured to generate sensor data indicative of contaminants in refuse material on the second conveyor. In some embodiments, the first contaminant sensor is configured to generate sensor data indicative of batteries in refuse material on the first conveyor. In some embodiments, the first contaminant sensor is configured to generate sensor data indicative of lithium-ion batteries in refuse material on the first conveyor. In some embodiments, the refuse vehicle includes a controller configured to receive data from the first contaminant sensor and to determine whether the data indicates a contaminant is present in a quantity of refuse.

Another exemplary embodiment of the present disclosure relates to a method of detecting contaminants in a quantity of refuse. The method includes receiving the quantity of refuse on a conveyor disposed within a refuse compartment of a refuse vehicle. The method also includes conveying the quantity of refuse from a first position within the refuse compartment to a second position within the refuse compartment. The method also includes receiving sensor data from a contaminant sensor configured to generate sensor data indicative of contaminants in refuse material being conveyed. The method also includes determining from the sensor data whether a contaminant is present in the quantity of refuse. Finally, the method includes initiating remedial action.

Another exemplary embodiment of the present disclosure relates to a refuse vehicle. The refuse vehicle includes a chassis, a cab coupled to the chassis, a refuse compartment supported by the chassis, a conveyor disposed within the refuse compartment, and a contaminant sensor disposed within the refuse compartment and oriented toward the conveyor. The refuse compartment defines a hopper volume and a storage volume. The conveyor is positioned to convey refuse received in the hopper volume to the storage volume. The contaminant sensor is configured to generate sensor data indicative of contaminants in refuse material on the conveyor.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

The disclosure will become more fully understood from the following detailed description, taken in conjunction with the accompanying figures, wherein like reference numerals refer to like elements, in which:

FIG. 1 is a perspective view of a front-loading refuse vehicle, according to an exemplary embodiment;

FIG. 2 is a side view of a rear-loading refuse vehicle, according to an exemplary embodiment;

FIG. 3 is a perspective view of a side-loading refuse vehicle, according to an exemplary embodiment;

FIG. 4 is a block diagram of a control system that may be used by any of the refuse vehicles of FIGS. 1-3, according to an exemplary embodiment;

FIG. 5 is a diagram illustrating a collection route for autonomous transport and collection by a refuse vehicle, according to an exemplary embodiment;

FIG. 6 is a cross-sectional side view of a refuse vehicle equipped with a contaminant detection system, according to an exemplary embodiment;

FIG. 7 is a cross-sectional side view of a refuse vehicle equipped with a contaminant detection system, according to another exemplary embodiment;

FIG. 8 is a cross-sectional side view of a refuse vehicle equipped with a contaminant detection system, according to still another exemplary embodiment;

FIG. 9 is a block diagram of a control system for a refuse vehicle, according to an exemplary embodiment; and

FIG. 10 is a flow diagram of a process for detecting the presence of contaminants received by a refuse vehicle, according to an exemplary embodiment.

### DETAILED DESCRIPTION

Before turning to the figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

### Overview

Refuse vehicles (e.g., a garbage truck, a waste collection truck, a sanitation truck, etc.) are vehicles configured to collect, process, and transport refuse. Hazardous articles or waste contaminants, such as batteries (e.g., lithium-ion batteries) are often disposed of with other refuse materials instead of being handled properly. When these contaminants are inadvertently collected and processed by a refuse vehicle not configured to do so, dangerous conditions can arise. For example, inadvertently compacting a lithium-ion battery intermixed with a quantity of refuse may cause a fire that is difficult to extinguish, and/or can ignite other materials that have been collected by the refuse vehicle, such as during waste compaction, transfer, and/or unloading operations.

Referring generally to the FIGURES, a refuse vehicle with a contaminant detection system for identifying hazardous and non-hazardous waste contaminants (e.g., batteries, abnormal and/or foreign materials, and/or materials of different types) is shown, according to various exemplary embodiments. The contaminant detection system includes one or more sensors configured to identify waste contaminants from refuse materials received by the refuse vehicle (e.g., to detect physical qualities of quantities of refuse that are consistent with the presence of contaminants). In some embodiments, the contaminant detection system includes a mechanism for enhancing contaminant detection from given quantities of waste material received by the refuse vehicle. For example, the contaminant detection system may include a conveyor system to distribute the waste materials received from a lift or grabber apparatus onto the refuse vehicle, and to prevent stacking and/or occlusion of waste contaminants within the refuse stream. The contaminant detection system may also include an apparatus or mechanism to isolate or contain identified contaminants in a safe manner for later disposal. Accordingly, the refuse vehicles described herein provide an improved means of collecting, processing, and transporting refuse that may contain contaminants or hazardous articles.

### Refuse Vehicle

### Front-loading Configuration

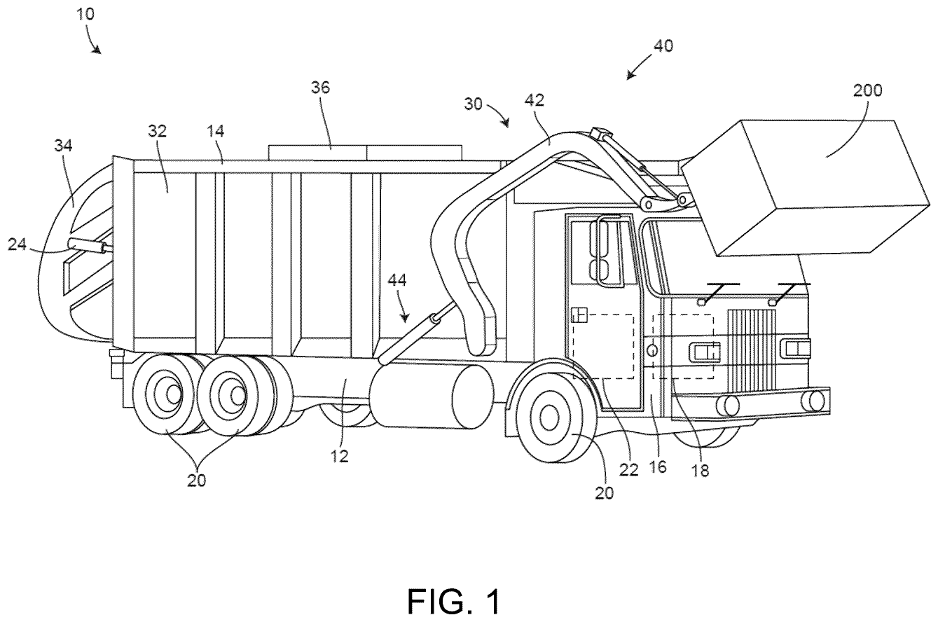

Referring to FIG. 1, a vehicle, shown as refuse vehicle 10 (e.g., a garbage truck, a waste collection truck, a sanitation truck, etc.), is shown that is configured to collect and store refuse along a collection route. In the embodiment of FIG. 1, the refuse vehicle 10 is configured as a front-loading refuse vehicle. The refuse vehicle 10 includes a chassis, shown as frame 12; a body assembly, shown as body 14, coupled to the frame 12 (e.g., at a rear end thereof, etc.); and a cab, shown as cab 16, coupled to the frame 12 (e.g., at a front end thereof, etc.). The cab 16 may include various components to facilitate operation of the refuse vehicle 10 by an operator (e.g., a seat, a steering wheel, hydraulic controls, a user interface, an acceleration pedal, a brake pedal, a clutch pedal, a gear selector, switches, buttons, dials, etc.). As shown in FIG. 1, the refuse vehicle 10 includes a prime mover, shown as engine 18, coupled to the frame 12 at a position beneath the cab 16. The engine 18 is configured to provide power to tractive elements, shown as wheels 20, and/or to other systems of the refuse vehicle 10 (e.g., a pneumatic system, a hydraulic system, etc.). The engine 18 may be configured to utilize one or more of a variety of fuels (e.g., gasoline, diesel, bio-diesel, ethanol, natural gas, etc.), according to various exemplary embodiments. The fuel may be stored in a tank 28 (e.g., a vessel, a container, a capsule, etc.) that is fluidly coupled with the engine 18 through one or more fuel lines.

According to an alternative embodiment, the engine 18 additionally or alternatively includes one or more electric motors coupled to the frame 12 (e.g., a hybrid refuse vehicle, an electric refuse vehicle, etc.). The electric motors may consume electrical power from any of an on-board storage device (e.g., batteries, ultra-capacitors, etc.), from an on-board generator (e.g., an internal combustion engine, etc.), or from an external power source (e.g., overhead power lines, etc.) and provide power to the systems of the refuse vehicle 10. The engine 18 may transfer output torque to or drive the wheels 20 of the refuse vehicle 10 through a transmission 22. The engine 18, the transmission 22, and one or more shafts, axles, gearboxes, etc., may define a driveline of the refuse vehicle 10.

According to an exemplary embodiment, the refuse vehicle 10 is configured to transport refuse from various waste receptacles within a municipality to a storage and/or processing facility (e.g., a landfill, an incineration facility, a recycling facility, etc.). As shown in FIG. 1, the body 14 includes a plurality of panels, shown as panels 32, a tailgate 34, and a cover 36. The panels 32, the tailgate 34, and the cover 36 define a collection chamber (e.g., hopper, etc.), shown as refuse compartment 30. Loose refuse may be placed into the refuse compartment 30 where it may thereafter be compacted. The refuse compartment 30 may provide temporary storage for refuse during transport to a waste disposal site and/or a recycling facility. In some embodiments, at least a portion of the body 14 and the refuse compartment 30 extend in front of the cab 16. According to the embodiment shown in FIG. 1, the body 14 and the refuse compartment 30 are positioned behind the cab 16. In some embodiments, the refuse compartment 30 includes a hopper volume and a storage volume. Refuse may be initially loaded into the hopper volume and thereafter transferred and/or compacted into the storage volume. According to an exemplary embodiment, the hopper volume is positioned forward of the cab 16 (e.g., refuse is loaded into a position of the refuse compartment 30 in front of the cab 16, a front-loading refuse vehicle, etc.). In other embodiments, the hopper volume is positioned between the storage volume and the cab 16 (e.g., refuse is loaded into a position of the refuse compartment 30 behind the cab 16 and stored in a position further toward the rear of the refuse compartment 30). In yet other embodiments, the storage volume is positioned between the hopper volume and the cab 16 (e.g., a rear-loading refuse vehicle, etc.).

The tailgate 34 may be hingedly or pivotally coupled with the body 14 at a rear end of the body 14 (e.g., opposite the cab 16). The tailgate 34 may be driven to rotate between an open position and a closed position by tailgate actuators 24. The refuse compartment 30 may be hingedly or pivotally coupled with the frame 12 such that the refuse compartment 30 can be driven to raise or lower while the tailgate 34 is open in order to dump contents of the refuse compartment 30 at a landfill. The refuse compartment 30 may include a packer assembly (e.g., a compaction apparatus) positioned therein that is configured to compact loose refuse.

Referring still to FIG. 1, the refuse vehicle 10 includes a first lift mechanism or system (e.g., a front-loading lift assembly, etc.), shown as lift assembly 40. The lift assembly 40 includes a pair of arms, shown as lift arms 42, coupled to at least one of the frame 12 or the body 14 on either side of the refuse vehicle 10 such that the lift arms 42 extend forward of the cab 16 (e.g., a front-loading refuse vehicle, etc.). The lift arms 42 may be rotatably coupled to frame 12 with a pivot (e.g., a lug, a shaft, etc.). The lift assembly 40 includes first actuators, shown as lift arm actuators 44 (e.g., hydraulic cylinders, etc.), coupled to the frame 12 and the lift arms 42. The lift arm actuators 44 are positioned such that extension and retraction thereof rotates the lift arms 42 about an axis extending through the pivot, according to an exemplary embodiment. Lift arms 42 may be removably coupled to a container, shown as refuse container 200 in FIG. 1. Lift arms 42 are configured to be driven to pivot by lift arm actuators 44 to lift and empty the refuse container 200 into the hopper volume for compaction and storage. The lift arms 42 may be coupled with a pair of forks or elongated members that are configured to removably couple with the refuse container 200 so that the refuse container 200 can be lifted and emptied. The refuse container 200 may be similar to the container attachment as described in greater detail in U.S. application Ser. No. 17/558,183, filed Dec. 12, 2021, the entire disclosure of which is incorporated by reference herein.

### Rear-Loading Configuration

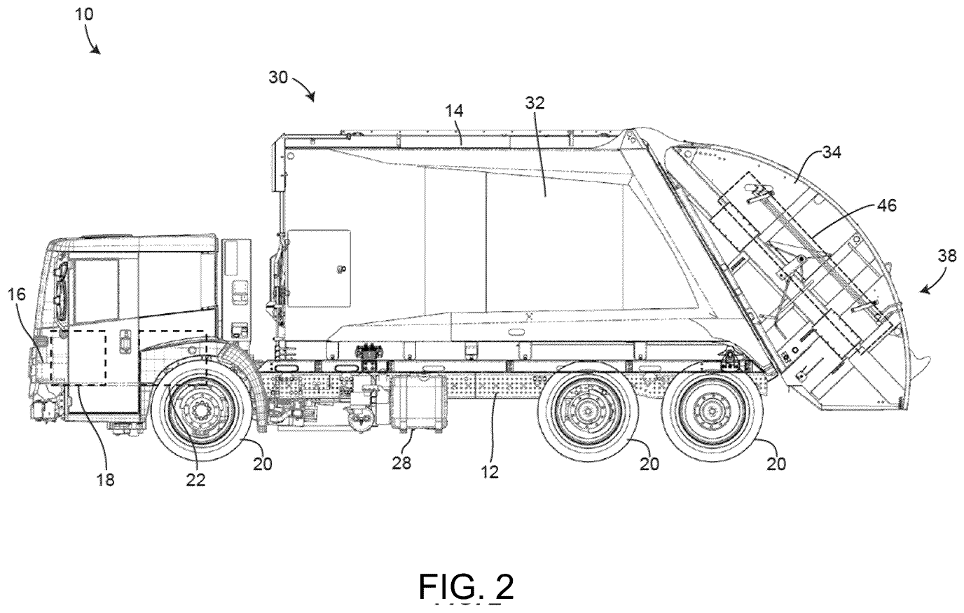

As shown in FIG. 2, the refuse vehicle 10 may be configured as a rear-loading refuse vehicle, according to some embodiments. In the rear-loading embodiment of the refuse vehicle 10, the tailgate 34 defines an opening 38 through which loose refuse may be loaded into the refuse compartment 30. The tailgate 34 may also include a packer 46 (e.g., a packing assembly, a compaction apparatus, a claw, a hinged member, etc.) that is configured to draw refuse into the refuse compartment 30 for storage. Similar to the embodiment of the refuse vehicle 10 described in FIG. 1 above, the tailgate 34 may be hingedly coupled with the refuse compartment 30 such that the tailgate 34 can be opened or closed during a dumping operation.

### Side-Loading Configuration

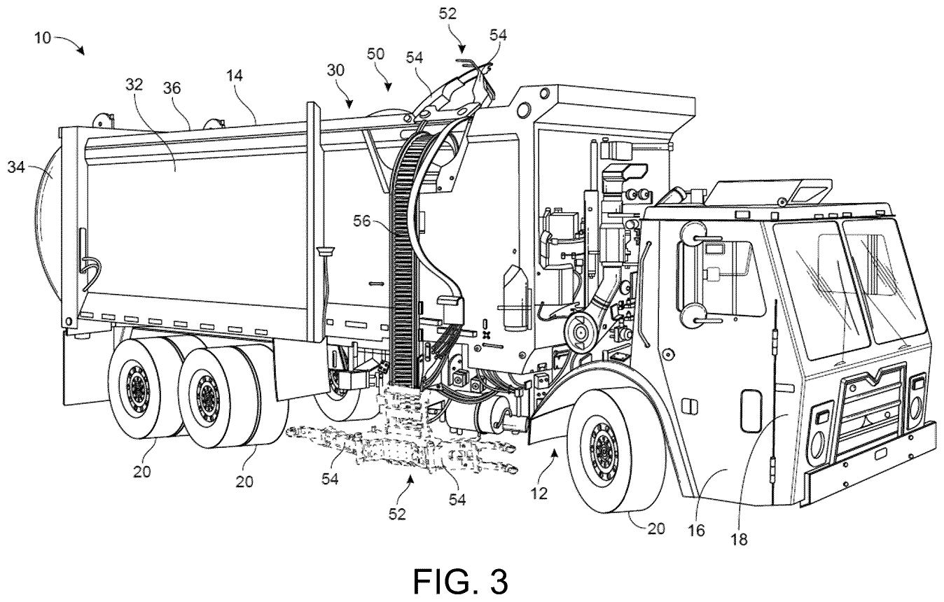

Referring to FIG. 3, the refuse vehicle 10 may be configured as a side-loading refuse vehicle (e.g., a zero radius side-loading refuse vehicle). The refuse vehicle 10 includes first lift mechanism or system, shown as lift assembly 50. Lift assembly 50 includes a grabber assembly, shown as grabber assembly 52, movably coupled to a track, shown as track 56, and configured to move along an entire length of track 56. According to the exemplary embodiment shown in FIG. 3, track 56 extends along substantially an entire height of body 14 and is configured to cause grabber assembly 52 to tilt near an upper height of body 14. In other embodiments, the track 56 extends along substantially an entire height of body 14 on a rear side of body 14. The refuse vehicle 10 can also include a reach system or assembly coupled with a body or frame of refuse vehicle 10 and lift assembly 50. The reach system can include telescoping members, a scissors stack, etc., or any other configuration that can extend or retract to provide additional reach of grabber assembly 52 for refuse collection.

Referring still to FIG. 3, grabber assembly 52 includes a pair of grabber arms shown as grabber arms 54. The grabber arms 54 are configured to rotate about an axis extending through a bushing. The grabber arms 54 are configured to releasably secure a refuse container to grabber assembly 52, according to an exemplary embodiment. The grabber arms 54 rotate about the axis extending through the bushing to transition between an engaged state (e.g., a fully grasped configuration, a fully grasped state, a partially grasped configuration, a partially grasped state) and a disengaged state (e.g., a fully open state or configuration, a fully released state/configuration, a partially open state or configuration, a partially released state/configuration). In the engaged state, the grabber arms 54 are rotated towards each other such that the refuse container is grasped therebetween. In the disengaged state, the grabber arms 54 rotate outwards such that the refuse container is not grasped therebetween. By transitioning between the engaged state and the disengaged state, the grabber assembly 52 releasably couples the refuse container with grabber assembly 52. The refuse vehicle 10 may pull up along-side the refuse container, such that the refuse container is positioned to be grasped by the grabber assembly 52 therebetween. The grabber assembly 52 may then transition into an engaged state to grasp the refuse container. After the refuse container has been securely grasped, the grabber assembly 52 may be transported along track 56 with the refuse container. When the grabber assembly 52 reaches the end of track 56, the grabber assembly 52 may tilt and empty the contents of the refuse container in refuse compartment 30. The tilting is facilitated by the path of the track 56. When the contents of the refuse container have been emptied into refuse compartment 30, the grabber assembly 52 may descend along the track 56 and return the refuse container to the ground. Once the refuse container has been placed on the ground, the grabber assembly may transition into the disengaged state, releasing the refuse container.

### Control System

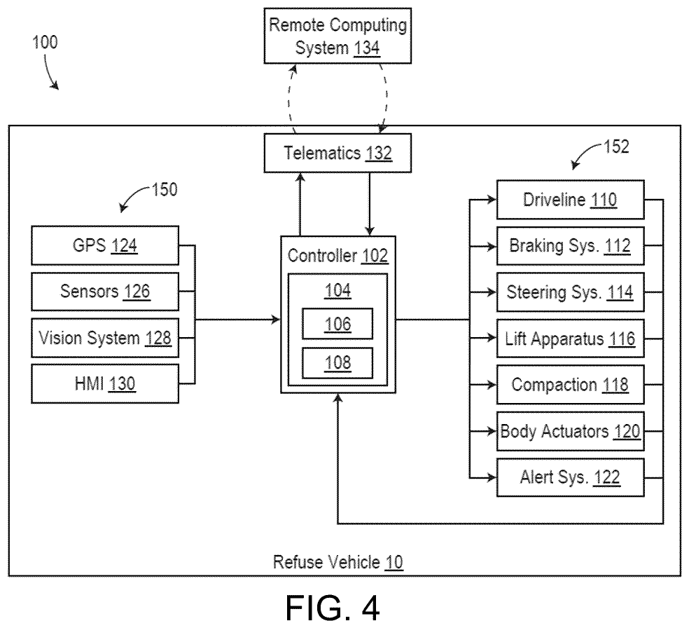

Referring to FIG. 4, the refuse vehicle 10 may include a control system 100 that is configured to facilitate operation of the refuse vehicle 10, or components thereof. In some embodiments, the control system 100 is configured to provide autonomous or semi-autonomous operation of the refuse vehicle 10, or components thereof. The control system 100 includes a controller 102 that is positioned on the refuse vehicle 10, a remote computing system 134, a telematics unit 132, one or more input devices 150, and one or more controllable elements 152. The input devices 150 can include a Global Positioning System (“GPS”), multiple sensors 126, a vision system 128 (e.g., an awareness system), and a Human Machine Interface (“HMI”). The controllable elements 152 can include a driveline 110 of the refuse vehicle 10, a braking system 112 of the refuse vehicle 10, a steering system 114 of the refuse vehicle 10, a lift apparatus 116 (e.g., the lift assembly 40, the lift assembly 50, etc.), a compaction system 118 (e.g., a packer assembly, the packer 46, etc.), body actuators 120 (e.g., tailgate actuators 24, lift or dumping actuators, etc.), and/or an alert system 122.

The controller 102 includes processing circuitry 104 including a processor 106 and memory 108. Processing circuitry 104 can be communicably connected with a communications interface of controller 102 such that processing circuitry 104 and the various components thereof can send and receive data via the communications interface. Processor 106 can be implemented as a general purpose processor, an application specific integrated circuit (ASIC), one or more field programmable gate arrays (FPGAs), a group of processing components, or other suitable electronic processing components.

Memory 108 (e.g., memory, memory unit, storage device, etc.) can include one or more devices (e.g., RAM, ROM, Flash memory, hard disk storage, etc.) for storing data and/or computer code for completing or facilitating the various processes, layers and modules described in the present application. Memory 108 can be or include volatile memory or non-volatile memory. Memory 108 can include database components, object code components, script components, or any other type of information structure for supporting the various activities and information structures described in the present application. According to some embodiments, memory 108 is communicably connected to processor 106 via processing circuitry 104 and includes computer code for executing (e.g., by at least one of processing circuitry 104 or processor 106) one or more processes described herein.

The controller 102 is configured to receive inputs (e.g., measurements, detections, signals, sensor data, etc.) from the input devices 150, according to some embodiments. In particular, the controller 102 may receive a GPS location from the GPS system 124 (e.g., current latitude and longitude of the refuse vehicle 10). The controller 102 may receive sensor data (e.g., engine temperature, fuel levels, transmission control unit feedback, engine control unit feedback, speed of the refuse vehicle 10, etc.) from the sensors 126. The controller 102 may receive image data (e.g., real-time camera data) from the vision system 128 of an area of the refuse vehicle 10 (e.g., in front of the refuse vehicle 10, rearwards of the refuse vehicle 10, on a street-side or curb-side of the refuse vehicle 10, at the hopper of the refuse vehicle 10 to monitor refuse that is loaded, within the cab 16 of the refuse vehicle 10, etc.). The controller 102 may receive user inputs from the HMI 130 (e.g., button presses, requests to perform a lifting or loading operation, driving operations, steering operations, braking operations, etc.).

The controller 102 may be configured to provide control outputs (e.g., control decisions, control signals, etc.) to the driveline 110 (e.g., the engine 18, the transmission 22, the engine control unit, the transmission control unit, etc.) to operate the driveline 110 to transport the refuse vehicle 10. The controller 102 may also be configured to provide control outputs to the braking system 112 to activate and operate the braking system 112 to decelerate the refuse vehicle 10 (e.g., by activating a friction brake system, a regenerative braking system, etc.). The controller 102 may be configured to provide control outputs to the steering system 114 to operate the steering system 114 to rotate or turn at least two of the wheels 20 to steer the refuse vehicle 10. The controller 102 may also be configured to operate actuators or motors of the lift apparatus 116 (e.g., lift arm actuators 44) to perform a lifting operation (e.g., to grasp, lift, empty, and return a refuse container). The controller 102 may also be configured to operate the compaction system 118 to compact or pack refuse that is within the refuse compartment 30. The controller 102 may also be configured to operate the body actuators 120 to implement a dumping operation of refuse from the refuse compartment 30 (e.g., driving the refuse compartment 30 to rotate to dump refuse at a landfill). The controller 102 may also be configured to operate the alert system 122 (e.g., lights, speakers, display screens, etc.) to provide one or more aural or visual alerts to nearby individuals.

The controller 102 may also be configured to receive feedback from any of the driveline 110, the braking system 112, the steering system 114, the lift apparatus 116, the compaction system 118, the body actuators 120, or the alert system 122. The controller may provide any of the feedback to the remote computing system 134 via the telematics unit 132. The telematics unit 132 may include any wireless transceiver, cellular dongle, communications radios, antennas, etc., to establish wireless communication with the remote computing system 134. The telematics unit 132 may facilitate communications with telematics units 132 of nearby refuse vehicles 10 to thereby establish a mesh network of refuse vehicles 10.

The controller 102 is configured to use any of the inputs from any of the GPS system 124, the sensors 126, the vision system 128, or the HMI 130 to generate controls for the driveline 110, the braking system 112, the steering system 114, the lift apparatus 116, the compaction system 118, the body actuators 120, or the alert system 122. In some embodiments, the controller 102 is configured to operate the driveline 110, the braking system 112, the steering system 114, the lift apparatus 116, the compaction system 118, the body actuators 120, and/or the alert system 122 to autonomously transport the refuse vehicle 10 along a route (e.g., self-driving), perform pickups or refuse collection operations autonomously, and/or transport to a landfill to empty contents of the refuse compartment 30. The controller 102 may receive one or more inputs from the remote computing system 134 such as route data, indications of pickup locations along the route, route updates, customer information, pickup types, etc. The controller 102 may use the inputs from the remote computing system 134 to autonomously transport the refuse vehicle 10 along the route and/or to perform the various operations along the route (e.g., picking up and emptying refuse containers, providing alerts to nearby individuals, limiting pickup operations until an individual has moved out of the way, etc.).

In some embodiments, the remote computing system 134 is configured to interact with (e.g., control, monitor, etc.) the refuse vehicle 10 through a virtual refuse truck as described in U.S. application Ser. No. 16/789,962, now U.S. Pat. No. 11,380,145, filed Feb. 13, 2020, the entire disclosure of which is incorporated by reference herein. The remote computing system 134 may perform any of the route planning techniques as described in greater detail in U.S. application Ser. No. 18/111,137, filed Feb. 17, 2023, the entire disclosure of which is incorporated by reference herein. The remote computing system 134 may implement any route planning techniques based on data received by the controller 102. In some embodiments, the controller 102 is configured to implement any of the cart alignment techniques as described in U.S. application Ser. No. 18/242,224, filed Sep. 5, 2023, the entire disclosure of which is incorporated by reference herein. The refuse vehicle 10 and the remote computing system 134 may also operate or implement geofences as described in greater detail in U.S. application Ser. No. 17/232,855, filed Apr. 16, 2021, the entire disclosure of which is incorporated by reference herein. Although various aspects of autonomous operation may be implemented by the controller 102, it should be understood that the controller 102 may also be configured to facilitate operation of the refuse vehicle by a vehicle operation, for example, based on operator inputs to the HMI 130.

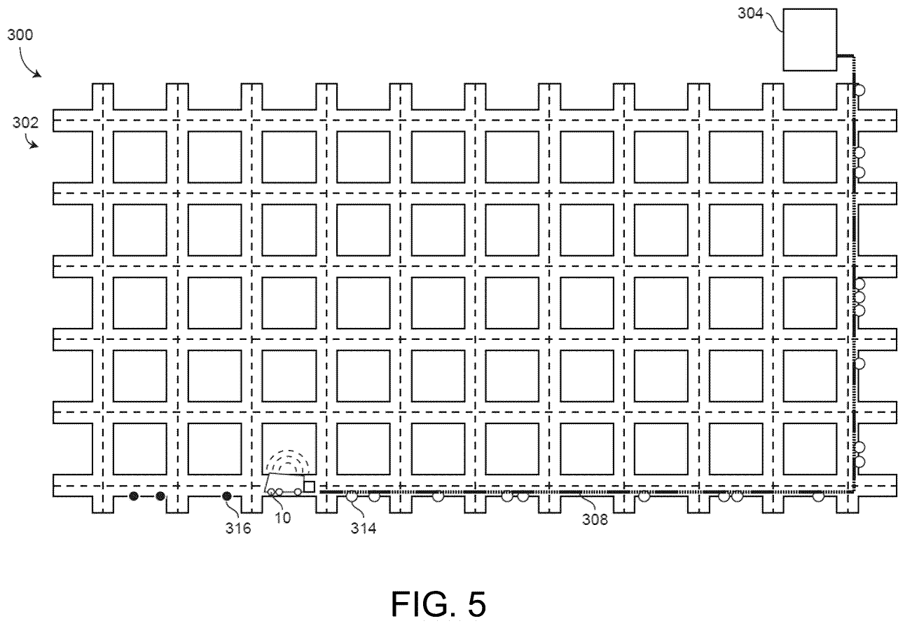

Referring to FIG. 5, a diagram 300 illustrates a route 308 through a neighborhood 302 for the refuse vehicle 10. The route 308 includes future stops 314 along the route 308 to be completed, and past stops 316 that have already been completed. The route 308 may be defined and provided by the remote computing system 134. The remote computing system 134 may also define or determine the future stops 314 and the past stops 316 along the route 308 and provide data regarding the geographic location of the future stops 314 and the past stops 316 to the controller 102 of the refuse vehicle 10. The refuse vehicle 10 may use the route data and the stops data to autonomously transport along the route 308 and perform refuse collection at each stop. The route 308 may end at a landfill 304 (e.g., an end location) where the refuse vehicle 10 may autonomously empty collected refuse, transport to a refueling location if necessary, and begin a new route.

### Contaminant Detection System

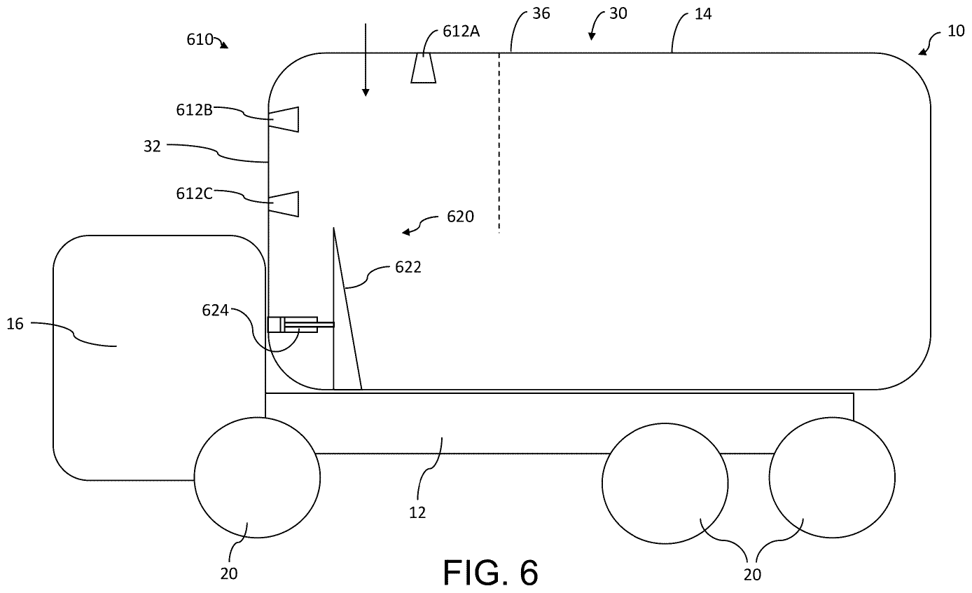

Referring to FIGS. 6-8, the refuse vehicle 10 may include a contaminant detection and/or separation system 610 to facilitate the detection, and subsequent separation and/or removal, of contaminants intermixed with refuse received into the refuse compartment 30 of the refuse vehicle 10. The contaminant may include hazardous materials such as batteries (e.g., lithium ion (Li-ion) batteries, lead-acid batteries, nickel-cadmium batteries (Ni—Cd), nickel-metal hydride batteries (Ni—MH), and/or other types of batteries). Although various contaminant detection methods and systems are described herein with respect to batteries, it should be appreciated that the same systems may be implemented to identify and/or separate various other types of contaminants. For example, and depending on the desired waste materials to be received by the refuse vehicle, the contaminant may include recyclable materials or non-recyclable materials, inorganic waste, chemical waste, and/or various other types of materials and objects.

The contaminant detection system 610 includes at least one contaminant sensor 612. The at least one contaminant sensor 612 is configured to generate sensor data and/or signals indicative of the presence of contaminants within a quantity of refuse and/or within view of the contaminant sensor 612. The sensor data of the at least one contaminant sensor 612 may be received as an input to a controller or control system (e.g., controller 102, a controller dedicated to the contaminant detection system 610, a remote controller, an edge computing device, etc.) for processing to determine if a contaminant is present within the waste stream received by the refuse vehicle and/or to perform remedial action if a contaminant is detected (e.g., isolating, separating, or containing contaminants or quantities of refuse containing contaminants).

The at least one contaminant sensor 612 may be selected to measure or detect qualities consistent with a specific type of contaminant (e.g., batteries). For example, the contaminant sensor 612 may be configured to identify a specific material that is indicative of the presence of a battery within the refuse stream (e.g., nickel, cadmium, lithium, etc.), or another material property that is indicative of the presence of a battery (e.g., size, shape, mass, density, etc.). The at least one contaminant sensor 612 may include an optical imaging sensor (e.g., a camera), a spectral imaging sensor (e.g., an x-ray, microwave, radio wave, gamma wave, or infrared sensor), a particle imaging sensor (e.g., a neutron, photon, or electron sensor), a magnetic field sensor (e.g., an electromagnetic sensor, a metal detector), or a gas analyzer.

In the embodiment of FIG. 6, the at least one contaminant sensor 612 is disposed within the refuse compartment 30 of the refuse vehicle 10 such that refuse material passes within the field of view of the at least one contaminant sensor 612 as the refuse material is received into, or moved about, the refuse compartment 30. In some embodiments, the contaminant detection system 610 is configured to facilitate repositioning the at least one contaminant sensor 612 (e.g., either manually or automatically) so that the refuse stream entering the refuse vehicle may be analyzed at different angles and/or positions.

In some embodiments, various ones of the contaminant sensor 612 may be positioned/orientated differently from one another within the refuse compartment 30 to measure qualities of a quantity of refuse at different angles or at different positions within the refuse compartment 30. In some embodiments, one or more of the at least one contaminant sensor 612 may be of a different type than one or more other of the at least one contaminant sensor 612. For example, a first contaminant sensor (e.g., 612A) of the at least one contaminant sensor 612 may be a magnetic field sensor and a second contaminant sensor (e.g., 612B) of the at least one contaminant sensor 612 may be an optical imaging sensor.

In an exemplary embodiment, and as shown in FIG. 6, the at least one contaminant sensor 612 is disposed within the hopper volume of the refuse compartment (i.e., the part of the refuse compartment 30 in which refuse is initially loaded) to measure the physical qualities of refuse as it is first received into the refuse compartment 30. For example, the at least one contaminant sensor 612 may be coupled to an inside surface of the cover 36 of the body 14, proximate the hopper volume of the refuse compartment 30 (e.g., 612A). The at least one contaminant sensor 612 may be coupled to an inside surface of one of the panels 32 of the body 14, proximate the hopper volume of the refuse compartment 30 (e.g., 612B and 612C). In some embodiments, one or more of the at least one contaminant sensor 612 may be coupled to the cover 36 and one or more other ones of the at least one contaminant sensor 612 may be coupled to one of the panels 32.

In some embodiments, the contaminant detection system 610 includes an agitation device (e.g., an excitation device, etc.). The agitation device may be a mechanical apparatus that disrupts or modifies the flow of refuse as it is received into the refuse compartment 30 (e.g., a deflector plate, a separator rod, a vibratory shaker, etc.) to distribute the refuse material across a surface or otherwise present the refuse to the at least one contaminant sensor 612 in a manner that improves the detection of qualities consistent with the presence of contaminants. In some embodiments, the agitation may crush, grind, or shred refuse received into the refuse compartment 30.

In some embodiments, the agitation device may be an excitation device that is configured to affect different materials in different ways to facilitate identification of various materials. For example, the agitation device may be a device that radiates (e.g., with microwaves) refuse material as the material is received into or moved about the refuse compartment 30 to enhance the detectability of the physical qualities consistent with the presence of contaminants. For example, the agitation device may include a microwave that radiates the refuse material passing therethrough to raise the temperature of specific types of contaminants or to otherwise affect a change in another physical quality of different contaminants within the refuse stream (e.g., to cause a chemical reaction of certain materials, etc.), so that the contaminant may be more easily detected using the one or more sensors.

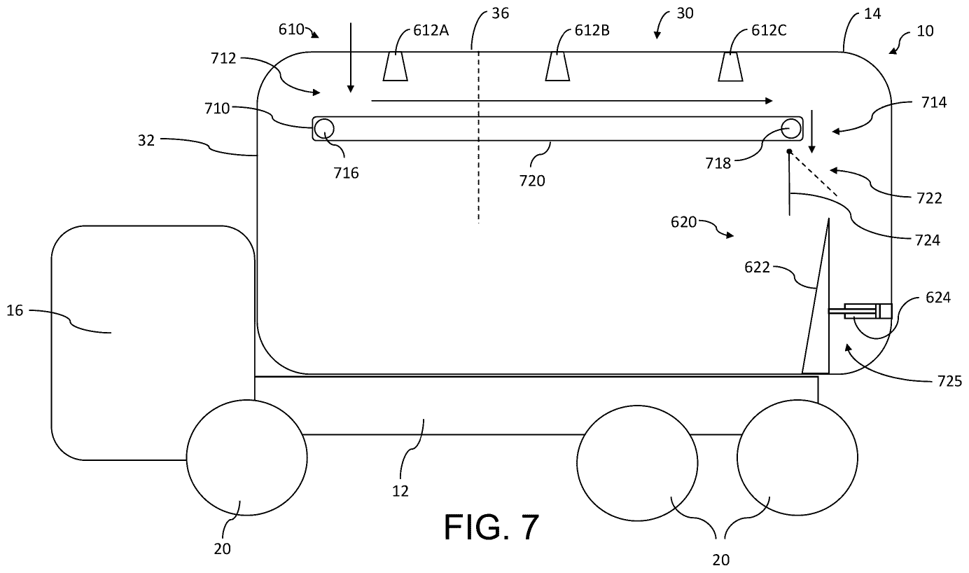

In an exemplary embodiment, and as shown in FIG. 7, the contaminant detection system 610 includes a first conveyor 710 to reduce the depth of refuse as the refuse is presented to the at least one contaminant sensor 612. The first conveyor 710 is disposed within the refuse compartment 30 defined by the body 14 of the refuse vehicle 10 and is positioned to convey refuse received at a first position 712 within the refuse compartment 30 to a second position 714 within the refuse compartment 30. As shown in FIG. 7, the first position 712 may be within the hopper volume of the refuse compartment 30 (i.e., the part of the refuse compartment 30 in which refuse is initially loaded). The second position 714 may be within the storage volume of the refuse compartment 30 (i.e., the part of the refuse compartment 30 in which refuse is stored and/or compacted after initial receipt into the refuse compartment 30). In some embodiments, the first position 712 may be within a front portion of the refuse compartment 30 (i.e., a portion of the refuse compartment proximate the cab 16) and the second position 714 may be within a rear portion of the refuse compartment 30 (i.e., a portion of the refuse compartment opposite the cab 16). In an exemplary embodiment, the first conveyor 710 is positioned within an upper portion of the refuse compartment 30 (i.e., nearer the cover 36 of the body 14 than the frame 12 of the refuse vehicle 10) and extends substantially parallel to the cover 36 and the frame 12.

The first conveyor 710 includes a first roller 716, a second roller 718, and a conveyor belt 720 looped around the first roller 716 and the second roller 718. At least one of the first roller 716 and the second roller 718 is driven to rotate by a motor (e.g., an electric motor, a pneumatic motor, a hydraulic motor, etc.). The rotation of the first roller 716 and/or the second roller 718 causes the conveyor belt 720 looped around the first roller 716 and the second roller 718 to move continuously about the first roller 716 and the second roller 718, allowing refuse received on the conveyor belt 720 to move from one position to another (e.g., from the first position 712 to the second position 714 within the refuse compartment 30). The first conveyor 710 may include additional rollers spaced between the first roller 716 and the second roller 718, and within the conveyor belt 720 looped around the first roller 716 and the second roller 718, to provide additional support to refuse received on the conveyor belt 720. The first conveyor 710 may include a tensioning device to ensure the conveyor belt 720 remains taut and aligned around the first roller 716 and the second roller 718, reducing slippage of the conveyor belt 720 and maintaining efficient operation of the first conveyor 710.

The conveyor belt 720 may be made of a fabric, an elastomer, or a composite material. The conveyor belt 720 may alternatively be made of metal components interlocked in a manner to create a belt of sufficient flexibility and strength. The conveyor belt 720 may have a plurality of raised ribs or protrusions along its outer surface to engage and separate refuse received on the conveyor belt 720. In some embodiments, the conveyor belt 720 defines a plurality of apertures (i.e., holes or perforations) to allow liquids received thereon to pass through the conveyor belt 720, or to sort out materials below a threshold size.

In some embodiments, the first conveyor 710 may include a shaker or some similar device configured to vibrate refuse materials received on the conveyor belt 720, causing the refuse material to separate and disperse along the conveyor belt 720. In some embodiments, the first conveyor 710 occupies substantially the width of the refuse compartment 30 (i.e., the distance between opposing panels 32 of the body 14) such that refuse received into the refuse compartment 30 through the cover 36 of the body 14 will be received on the first conveyor 710, and to increase a surface area for distribution of the refuse material. In other embodiments, refuse received into the refuse compartment 30 of the refuse vehicle 10 is directed onto the first conveyor 710 by a trough, flue, or other conduit.

As shown in FIG. 7, the at least one contaminant sensor 612 may be positioned above the first conveyor 710 to measure physical qualities of the refuse that may indicate the presence of contaminants intermixed with the refuse as the refuse is conveyed from the first position 712 to the second position 714 on the first conveyor 710. In some embodiments, each of the at least one contaminant sensor 612 is positioned at a different location above the first conveyor 710 (see, e.g., 612A, 612B, and/or 612C) to observe respective portions of the first conveyor 710. In other embodiments, different types of contaminant sensors 612 may be co-located in the same or similar positions along the refuse vehicle to determine different properties of the waste stream passing thereby.

The at least one contaminant sensor 612 may be coupled to an inside surface of the cover 36 of the body 14 of the refuse vehicle 10. In some embodiments, the at least one contaminant sensor 612 is coupled to the cover 36 in a manner that allows the position or angle of the at least one contaminant sensor 612 to be manually or controllably adjusted relative to the first conveyor 710. In an exemplary embodiment, a first contaminant sensor (e.g., 612A) of the at least one contaminant sensor 612 is a magnetic field sensor, and a second contaminant sensor (e.g., 612B) of the at least one contaminant sensor 612 is an optical imaging sensor, a thermal imaging sensor, or another type of contaminant sensor.

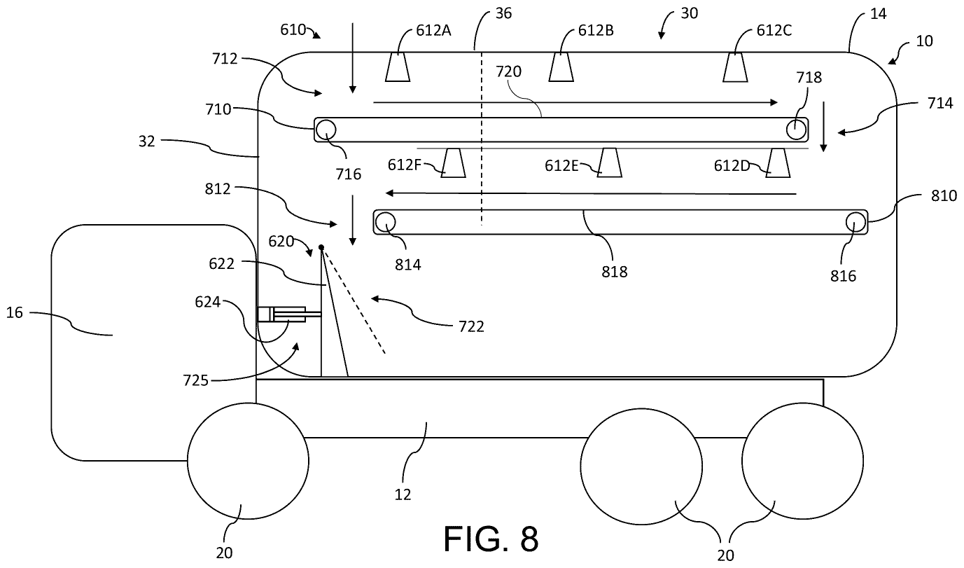

In an exemplary embodiment, and as shown in FIG. 8, the contaminant detection system 610 includes a second conveyor 810 to further agitate refuse received into the refuse compartment 30 of the refuse vehicle 10 and to provide greater scanning area for the contaminant detection system 610 to detect contaminants intermixed with the refuse. The second conveyor 810 is disposed within the refuse compartment 30 and positioned to convey refuse deposited by the first conveyor 710 at the second position 714 within the refuse compartment 30 to a third position 812 within the refuse compartment 30. In some embodiments, where the second position 714 is within a rear portion of the refuse compartment 30 (i.e., a portion of the refuse compartment opposite the cab 16), the third position 812 is within a front portion of the refuse compartment 30 (i.e., a portion of the refuse compartment proximate the cab 16). In an exemplary embodiment, the second conveyor 810 is positioned below the first conveyor 710 and extends substantially parallel to the first conveyor 710.

The second conveyor 810 may be similar in construction to the first conveyor 710. As shown in FIG. 8, the second conveyor 810 includes a first roller 814, a second roller 816, and a conveyor belt 818 looped around the first roller 814 and the second roller 816. At least one of the first roller 814 and the second roller 816 is driven to rotate by a motor (e.g., an electric motor, a pneumatic motor, a hydraulic motor, etc.). The rotation of the first roller 814 or the second roller 816 causes the conveyor belt 818 looped around the first roller 814 and the second roller 816 to move continuously about the first roller 814 and the second roller 816, allowing refuse received on the belt to move from one position to another (e.g., from the second position 714 to the third position 514 within the refuse compartment 30). The second conveyor 810 may include additional rollers spaced between the first roller 814 and the second roller 816, and within the conveyor belt 818 looped around the first roller 814 and the second roller 816, to provide additional support to refuse received on the conveyor belt 818. The second conveyor 810 may include a tensioning device to ensure the conveyor belt 818 remains taut and aligned around the first roller 814 and the second roller 816, reducing slippage of the conveyor belt 818 and maintaining efficient operation of the second conveyor 810.

The conveyor belt 818 may be made of a fabric, an elastomer, a composite material, and/or another type of conveyor belt material. The conveyor belt 818 may alternatively be made of metal components interlocked in a manner to create a belt of sufficient flexibility and strength. The conveyor belt 818 may have a plurality of raised ribs or protrusions along its outer surface to engage and separate refuse received on the conveyor belt. In some embodiments, the conveyor belt 818 defines a plurality of apertures (i.e., holes or perforations) to allow liquids and/or materials below a threshold size to pass through the conveyor belt 818 and into another portion of the refuse compartment. The second conveyor 810 may include a shaker or some similar device configured to vibrate refuse materials received on the conveyor belt 818, causing the refuse material to separate and disperse along the conveyor belt 818. In some embodiments, the second conveyor 810 occupies substantially the width of the refuse compartment 30 (i.e., the distance between opposing panels 32 of the body 14) such that refuse deposited by the first conveyor 710 will be received on the second conveyor 810, and/or to increase the overall surface area to distribute the refuse materials. In other embodiments, refuse deposited by the first conveyor 710 is directed onto the second conveyor 810 by a trough, flue, or other conduit.

As shown in FIG. 8, at least one contaminant sensor (e.g., 612D, 612E, or 612F) may be positioned above the second conveyor 810 to measure physical qualities of the refuse that may indicate the presence of contaminants intermixed with the refuse as the refuse is conveyed from the second position 714 to the third position 812 on the second conveyor 810. In some embodiments, multiple contaminant sensors (e.g., 612D, 612E, and/or 612F) of the at least one contaminant sensor 612 are position above the second conveyor 810. In some embodiments, the at least one contaminant sensor, or the multiple contaminant sensors, of the at least one contaminant sensor 612, positioned above the second conveyor 810, is coupled to an inside surface of one of the panels 32 of the body 14. The at least one contaminant sensor, or the multiple contaminant sensors, of the at least one contaminant sensor 612, positioned above the second conveyor 810, may be mounted in a manner that allows the position or angle of the at least one contaminant sensor, or the multiple contaminant sensors, of the at least one contaminant sensor 612, positioned above the second conveyor 810, to be manually or controllably adjusted relative to the second conveyor 810. In an exemplary embodiment, a first contaminant sensor (e.g., 612F) of the at least one contaminant sensor 612 is a magnetic field sensor, and a second contaminant sensor (e.g., 612G) of the at least one contaminant sensor 612 is an optical imaging sensor, a thermal imaging sensor, or another type of contaminant sensor.

It should be understood that the position of the contaminant sensors relative to the conveyor may be different in various embodiments. The at least one contaminant sensor 612 may be positioned anywhere within the refuse compartment 30 such that refuse received into the refuse compartment 30, or processed within the refuse compartment 30, passes within a field of view of the at least one contaminant sensor 612. In some embodiments, the at least one contaminant sensor 612 is coupled to an inside surface of a sidewall of the body 14. In some embodiments, the at least one contaminant sensor 612 is coupled to an inside surface of an end wall of the body 14. In some embodiments, the at least one contaminant sensor 612 is coupled to the first conveyor 710 or the second conveyor 810.

In some embodiments, the at least one contaminant sensor 612 includes a transmitter (e.g., an x-ray transmitter, a neutron transmitter, etc.) and a receiver (e.g., an x-ray receiver, a neutron receiver, etc.), the transmitter and the receiver configured to function cooperatively. In such embodiments, the transmitter and receiver of the at least one contaminant sensor 612 are positioned within the refuse compartment 30 and relative to the first conveyor 710 and/or the second conveyor 810 to function cooperatively. For example, the transmitter of the at least one contaminant sensor 612 may be positioned above a portion of the first conveyor 710 and/or the second conveyor 810, and the receiver of the at least one contaminant sensor 612 may be positioned below the same portion of the first conveyor 710 and/or the second conveyor 810 to function cooperatively with the transmitter of the at least one contaminant sensor 612.

In an exemplary embodiment, and as shown in FIGS. 7 and 8, the contaminant detection system 610 includes a contaminant separating mechanism 722 to separate and/or isolate individual contaminants, or quantities of refuse containing contaminants. Referring to FIG. 7, in some embodiments, the contaminant separating mechanism 722 includes a deflector plate 724. The deflector plate 724 is selectively movable between a first position and a second position that is offset from the first position (e.g., rotationally offset). In some embodiments, when the deflector plate 724 is in the first position, refuse received into the refuse compartment 30 is directed into a storage volume of the refuse compartment 30 to be stored and/or compacted for later delivery to a waste disposal site and/or a recycling facility. When the deflector plate 724 is in the second position, refuse received into the refuse compartment 30 is directed into a containment volume 725 of the refuse compartment 30 to be isolated from the other refuse stored within the refuse compartment 30 to be safely stored and removed. The deflector plate 724 may be positioned below the first conveyor 710 (see, e.g., FIG. 7) or the second conveyor 810, to direct the flow of refuse as it exits the first conveyor 710 or the second conveyor 810.

The containment volume 725 may be partially filled, or selectively fillable, with a non-flammable material (e.g., sand, foam, water, a class D fire suppressant agent etc.) to prevent isolated contaminants from igniting or to prevent the spread of a fire from the containment volume 725 to the storage volume. For example, the contaminant detection system 610 may include a remediation system that is configured to dispense a contaminant isolating and/or neutralizing agent into the containment volume 725 (e.g., in response or subsequent to detecting a contaminant within refuse material and directing the refuse material into the containment volume 725 by actuating the contaminant separating mechanism 722). In some embodiments, the containment volume 725 may be defined, at least in part, by a thermally resistant material (e.g., a ceramic, a high temperature metal, a material coated with a fire-resistant coating, etc.).

In some embodiments, as shown in FIGS. 6-8, the refuse vehicle 10 includes a packing assembly 620. The packing assembly 620 is disposed within the refuse compartment and configured to compact refuse stored therein and/or to eject refuse therefrom (e.g., through the tailgate 34). The packing assembly 620 includes a packing plate 622 (e.g., a pack and/or eject panel) and a pack actuator 624. The packing plate 622 is configured to move longitudinally through the refuse compartment 30 between a retracted position and an extended position, interacting with, and compacting against a part of the body 14, refuse stored within the refuse compartment 30. The pack actuator 624 is a linear actuator (e.g., a hydraulic cylinder, an electric linear actuator, etc.) coupled to the packing plate 622 at a first end and to a part of the body 14 at a second end such that when the actuator is extended or retracted, the packing plate 622 moves between the retracted position and the extended position. The pack actuator 624 may be controllably operated by a controller (e.g., controller 102). In some embodiments, the packing assembly 620 is disposed within a front portion of the refuse compartment 30 (see, e.g., FIGS. 6 and 8) so as to compact refuse stored within the refuse compartment 30 towards a rear portion of the refuse compartment 30. In other embodiments, the packing assembly 620 is disposed within a rear portion of the refuse compartment 30 (see, e.g., FIG. 7) so as to compact refuse stored within the refuse compartment 30 towards a front portion of the refuse compartment 30. In some embodiments, the packing assembly 620 at least partially defines the containment volume 725 (e.g., the packing plate 622 may divide the refuse compartment into the storage volume and the containment volume 725).

In some embodiments, and as shown in FIG. 8, the contaminant separating mechanism 722 is coupled, or integral, to the packing assembly 620. In an exemplary embodiment, a part of the packing plate 622 may be rotatably coupled to the rest of the packing plate 622 and movable between a first position and a second position. When the rotatable part of the packing plate 622 is in the second position, the packing plate 622 may be extended, by the pack actuator 624, partially between the retracted position and the extended position before the rotatable part of the packing plate 622 is moved to the first position, to isolate (e.g., to sweep) a quantity of refuse from the other refuse stored within the refuse compartment. The pack actuator 624 may then retract the packing plate 622 to the retracted position (e.g., to slide) to move the isolated quantity of refuse into the containment volume.

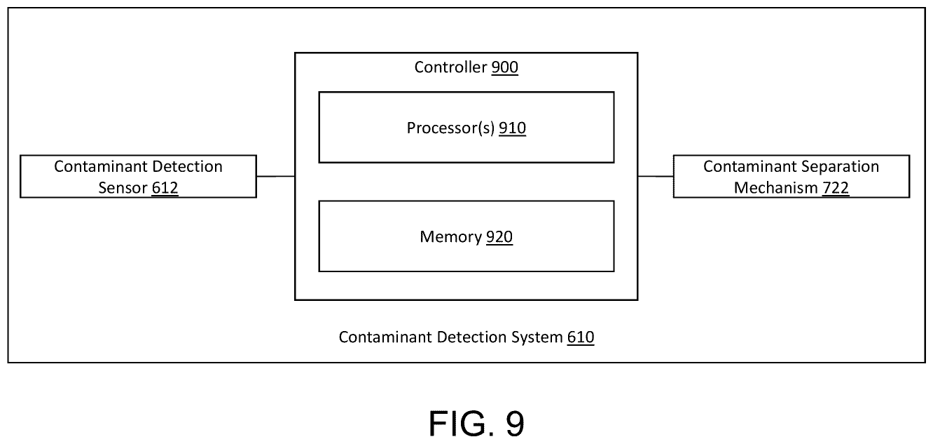

In some embodiments, and as shown in FIG. 9, the contaminant detection system 610 includes a controller, shown as contaminant detection system controller 900. The contaminant detection system controller 900 includes one or more processor 910 operatively connected and configured to execute instructions stored on a memory 920. The one or more processor 910 can be implemented as a general-purpose processor, an application specific integrated circuit (ASIC), one or more field programmable gate arrays (FPGAs), a group of processing components, or other suitable electronic processing components.

The memory 920 (e.g., memory, memory unit, storage device, etc.) can include one or more devices (e.g., RAM, ROM, flash memory, hard disk storage, etc.) for storing data and/or computer code for completing or facilitating the various processes, layers and modules described in the present application. The memory 920 can be or include volatile memory or non-volatile memory. The memory 920 can include database components, object code components, script components, or any other type of information structure for supporting the various activities and information structures described in the present application. According to some embodiments, memory 920 is communicably connected to the one or more processor 910 and includes computer code for executing one or more processes described herein.

The contaminant detection system controller 900 is configured to receive data from the at least one contaminant sensor 612. In some embodiments, the contaminant detection system controller 900 is configured to operatively control the contaminant separating mechanism 722. In some embodiments the contaminant detection system controller 900 is implemented on, or constitutes a subcomponent of, controller 102. In other embodiments, the contaminant detection system controller 900 is a standalone controller, dedicated to the operation of the contaminant detection system 610.

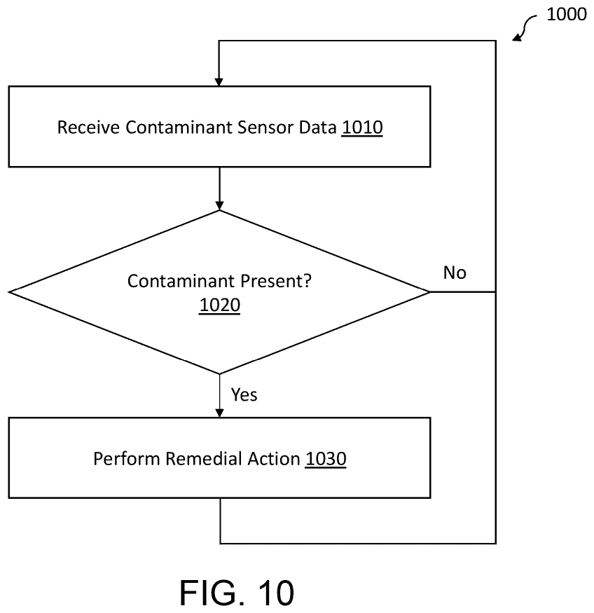

Referring to FIG. 10, a flow diagram of a method 1000 for identifying and/or isolating contaminants intermixed with refuse material received into the refuse compartment 30 of a refuse vehicle is provided, according to an exemplary embodiment. The method 1000 may be implemented on the controller 102 or the contaminant detection system controller 900 and will therefore be described with reference to FIGS. 4 and 9. In other embodiments, the method 1000 may include additional, fewer, and/or different operations.

The method 1000 includes the step of receiving contaminant sensor data, at 1010. In some embodiments, operation 1010 includes receiving, by the controller 900, from the at least one contaminant sensor, sensor data indicative of the presence of contaminants within the refuse material passing across the field of view of the at least one contaminant sensor. The contaminant sensor data may be in the form of digital or analog electrical signals. Operation 1010 may include storing the contaminant sensor data in memory (e.g., memory 108 or memory 920) for subsequent processing, and/or transmission to a cloud or other remote computing device (e.g., a fleet management service, etc.).

The method 1000 includes the step of determining the presence of contaminants, at 1020. In some embodiments, data received at operation 1010 is analyzed by the controller to determine whether the data indicates the presence of contaminants in the refuse material to which the data corresponds. In an exemplary embodiment, where one of the at least one contaminant sensor 612 is a magnetic field sensor, and another one of the at least one contaminant sensor 612 is an imaging sensor, operation 1020 may include first analyzing the data from the magnetic field sensor for data indicative of the presence of certain metals. If certain metals are detected in a quantity of refuse, the data from the imaging sensor corresponding to the same quantity of refuse may be analyzed (e.g., by computer vision methods, a machine learning model, artificial intelligence, etc.) to determine the existence of a particular type of contaminant (e.g., batteries such as lithium-ion batteries).

In some embodiments, operation 1020 includes exciting or agitating refuse material in a field of view of the at least one contaminant sensor 612, monitoring properties of the refuse material while the refuse material is being excited or agitated, and determining, based on changes in the properties of the refuse material, whether a contaminant is present in the refuse material. For example, operation 1020 may include activating, by the controller, a microwave emitter to excite refuse material within the field of view of a thermal sensor, monitoring thermal data output by the thermal sensor while the refuse material is being excited with microwaves, and determining, based on changes in the thermal data during the excitation, whether a contaminant is present in the refuse material.

In some embodiments, operation 1020 includes analyzing, by the controller, data from a first one of the at least one contaminant sensor 612 for an indication that a contaminant is present within the field of view of the first of the at least one contaminant sensor 612, disabling one or more function of the refuse vehicle 10 (e.g., the first conveyor 710, the second conveyor 810, the packing assembly 620, a lift mechanism, etc.) until the presence of the contaminant is confirmed and/or remedied by the operator of the refuse vehicle 10 or a remote party. For example, if sensor data satisfies a threshold condition indicative of a contaminant, operation 1020 may image the refuse satisfying the threshold condition and transfer the image to the operator of the refuse vehicle 10 or to a cloud service. Operation 1020 may disable one or more function of the refuse vehicle 10 until confirmation of the presence of a contaminant is received and/or remedial action is taken (e.g., removing or isolating the identified contaminant). A part of operation 1020 may be performed by a remote computer system (e.g. remote computing system 134, an edge computing device, etc.).

In some embodiments, operation 1020 includes adjusting, by the controller, one or more operating parameter (e.g., power, wavelength, frequency, intensity, etc.) of the at least one contaminant sensor 612. In some embodiments, operation 1020 includes repositioning, by the controller, the one or more contaminant sensor 612.

When contaminants are detected at operation 1020, the method 1000 includes the step of performing remedial action, at 1030. In some embodiments, operation 1030 includes actuating, by the controller, the contaminant separating mechanism 722 to isolate the contaminant or refuse material containing the contaminant from other refuse in the refuse compartment 30 of the refuse vehicle 10. In some embodiments, operation 1030 includes activating, by the controller, an alarm or indicator to allow the operator of the refuse vehicle 10 to take remedial action (i.e., manually removing or isolating the detected contaminant). In some embodiments, operation 1030 includes disabling, by the controller, one or more component of the refuse vehicle 10 (e.g., the first conveyor 710, the second conveyor 810, the packing assembly 620, a lift apparatus, etc.) until the detected contaminant is removed.

In some embodiments, operation 1030 includes automatically routing or rerouting the refuse vehicle 10 in response to determining a contaminant is present, at operation 1020. Upon detection of a contaminant, the controller 900 may initiate an updated route plan that diverts the refuse vehicle 10 from its standard collection path. In some embodiments, the rerouting may account for factors such as the type and severity of the contaminant, regulatory handling requirements, geographic proximity of various facilities, and refuse vehicle 10 operating constraints (e.g., remaining capacity, fuel levels, time-of-day, etc.). Based on these considerations, the controller 900 may direct the refuse vehicle 10 to a designated facility capable of safely processing, neutralizing, or disposing of the specific contaminant identified.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

References herein to a “remote computing system,” a “cloud,” or a “cloud computing system,” may include a computing system, or network of computing systems, having an edge computing device.

As utilized herein with respect to numerical ranges, the terms “approximately,” “about,” “substantially,” and similar terms generally mean +/-10% of the disclosed values. When the terms “approximately,” “about,” “substantially,” and similar terms are applied to a structural feature (e.g., to describe its shape, size, orientation, direction, etc.), these terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims. It is noted that the terms “exemplary” and “example” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like, as used herein, mean the joining of two members directly or indirectly to one another. Such joining may be stationary (e.g., permanent, etc.) or moveable (e.g., removable, releasable, etc.). Such joining may be achieved with the two members or the two members and any additional intermediate members being integrally formed as a single unitary body with one another or with the two members or the two members and any additional intermediate members being attached to one another.

References herein to the positions of elements (e.g., “top,” “bottom,” “above,” “below,” “between,” etc.) are merely used to describe the orientation of various elements in the figures. It should be noted that the orientation of various elements may differ according to other exemplary embodiments, and that such variations are intended to be encompassed by the present disclosure.

Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

It is important to note that the construction and arrangement of the systems as shown in the exemplary embodiments is illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements. It should be noted that the elements and/or assemblies of the components described herein may be constructed from any of a wide variety of materials that provide sufficient strength or durability, in any of a wide variety of colors, textures, and combinations. Accordingly, all such modifications are intended to be included within the scope of the present inventions. Other substitutions, modifications, changes, and omissions may be made in the design, operating conditions, and arrangement of the preferred and other exemplary embodiments without departing from scope of the present disclosure or from the spirit of the appended claims.

1. A refuse vehicle, comprising:
a chassis;
a body supported by the chassis, the body defining a refuse compartment and a containment volume;
a contaminant sensor disposed within the refuse compartment;
a conveyor disposed within the refuse compartment and configured to convey refuse material past the contaminant sensor;
a contaminant separating mechanism configured to selectively direct refuse material into the containment volume; and
a controller configured to:
receive sensor data from the contaminant sensor;
determine a presence of a contaminant within the refuse material based on the sensor data; and
actuate the contaminant separating mechanism in response to the presence of the contaminant to direct the refuse material containing the contaminant into the containment volume.

2. The refuse vehicle of claim 1, further comprising at least one of sand, foam, water, or a class D fire suppressant agent within the containment volume.

3. The refuse vehicle of claim 1, wherein the contaminant sensor is one of a camera, a spectral imaging sensor, a particle imaging sensor, or a magnetic field sensor.

4. The refuse vehicle of claim 1, further comprising:
a second contaminant sensor disposed within the refuse compartment and configured to detect a contaminant in refuse material; and
a second conveyor configured disposed within the refuse compartment and configured to convey refuse material past the second contaminant sensor.

5. The refuse vehicle of claim 1, further comprising an excitation device configured to agitate or radiate refuse material to enhance detectability of contaminants by the contaminant sensor.

6. The refuse vehicle of claim 1, wherein the controller is further configured to disable a compaction system or a lift mechanism upon determining the presence of the contaminant.

7. The refuse vehicle of claim 1, wherein the contaminant separating mechanism comprises a deflector plate selectively movable to direct refuse material containing the contaminant into the containment volume.

8. The refuse vehicle of claim 7, further comprising a packer assembly disposed within storage volume and actuatable to compact refuse material therein;
wherein, the deflector plate is integrated with a packing plate of the packer assembly.

9. A contaminant detection system, comprising:
a refuse body defining a storage compartment therein;
a contaminant sensor configured to generate sensor data indicative of a contaminant within refuse deposited into the storage compartment; and
a controller configured to:
receive sensor data from the contaminant sensor;
determine a presence of a contaminant within refuse deposited into the storage compartment based on the sensor data; and
initiating remedial action in response to the presence of the contaminant to isolate or remove the contaminant from the storage compartment.

10. The contaminant detection system of claim 9, further comprising a conveyor disposed within the storage compartment and configured to convey the refuse deposited into the storage compartment past the contaminant sensor.

11. The contaminant detection system of claim 9, further comprising a second contaminant sensor disposed within the storage compartment and configured to detect a contaminant within the refuse deposited into the storage compartment; and
a second conveyor disposed within the storage compartment and configured to convey the refuse deposited into the storage compartment past the second contaminant sensor.

12. The contaminant detection system of claim 9, further comprising a contaminant separating mechanism configured to selectively direct refuse containing a contaminant into a containment volume defined within the refuse body.

13. The contaminant detection system of claim 12, wherein the contaminant separating mechanism comprises a deflector plate selectively movable to direct refuse material containing the contaminant into the containment volume.

14. The contaminant detection system of claim 13, further comprising a packer assembly disposed within storage volume and actuatable to compact refuse material therein;
wherein, the deflector plate is integrated with a packing plate of the packer assembly.

15. The contaminant detection system of claim 9, further comprising an excitation device configured to agitate or radiate refuse material to enhance detectability of contaminants by the contaminant sensor.

16. A method of detecting contaminants in refuse material within a refuse storage compartment, the method comprising:
receiving, by a controller, sensor data from a contaminant sensor;
determining, by the controller based on the sensor data, that a contaminant is present in the refuse material; and
initiating, by the controller in response to the presence of the contaminant, remedial action to at least one of isolate the contaminant or remove the contaminant from the refuse storage compartment.

17. The method of claim 16, further comprising actuating, by the controller, a conveyor to convey refuse received into the refuse storage compartment past the contaminant sensor.

18. The method of claim 16, wherein initiating remedial action includes actuating, by the controller, a refuse contaminant separation mechanism configured selectively direct refuse containing a contaminant into a containment volume defined within the refuse storage compartment.

19. The method of claim 16, further comprising activating, by the controller, an excitation device configured to agitate or radiate refuse material to enhance detectability of contaminants by the contaminant sensor.

20. The method of claim 16, wherein initiating remedial action includes disabling, by the controller, one of a compaction system or a lift mechanism upon determining the presence of the contaminant.
